(19) United States
(12) Patent Application Publication
(10) Pub. No.: US 2007/0196877 A1
(43) Pub. Date: Aug. 23, 2007

(54) Rapid, low-cost assay for detecting brettanomyces and other spoilage yeast in wine

(76) Inventors: Michael O'Neill, Yorba Linda, CA (US); Stewart Lebrun, Anaheim Hills, CA (US)

Correspondence Address: MICHAEL A. SHIPPEY, PH. D., 4848 LAKEVIEW AVENUE, SUITE B, YORBA LINDA, CA, 92886, US

(21) Appl. No.: 11/601,169

(22) Filed: Nov. 17, 2006

Related U.S. Application Data
(60) Provisional application No. 60/737,787, filed on Nov. 17, 2005.

(51) Int. Cl. G01N 33/569; C12M 3/00

(52) U.S. Cl. 435/7.31; 435/287.2

(57) ABSTRACT

In one aspect, the present invention relates to a rapid, sensitive and cost-effective immunoassay for the detection of Brettanomyces yeast in wine.

### PRIORITY NOTICE

This application claims priority from the provisional patent of the same title, and common inventorship, filed on Nov. 17, 2005, and of Ser. No. 60/737,787.

### BACKGROUND OF THE INVENTION

1. Field of the Invention

In one aspect, the present invention relates to a rapid, sensitive and cost-effective immunoassay for the detection of Brettanomyces yeast in wine.

2. Description of the Related Art

Spoilage of wines by Brettanomyces (“Brett”) yeast is a growing issue, due in part to currently popular winemaking practices such as extended “hang time”, post-fermentation maceration, barrel ageing, and a desire to reduce the use of SO₂. Brett is also on the increase because of the current trend for ‘natural’ wines and a move towards ‘international’ styles of red wine, made in an extracted style from super-ripe grapes. Brett grows in finished wine, especially wine aged in barrels, often producing undesirable odors. The sporulating form of this yeast is known as Dekkera. Brett is a slow-growing yeast, unlike the fermentation yeast, Saccharomyces cerevisiae, but it is a very tenacious yeast, being very difficult to eliminate from a winery once it becomes established. Some strains of Brett, as it is known in the wine industry, grow in a mold-like way. Their hair-like hyphae can penetrate into the surfaces of oak barrels. This is the reason for the caveat in the wine industry not to buy used barrels—they may have become infected with Brett. Brett is widespread, and virtually every barrel of red wine has the potential to go ‘bretty’, and this represents an increasing problem even in new world countries such as Australia. Once Brett gets established in a winery it is difficult to keep it from contaminating wine. Brett contamination may occurs in pipes, pumps, pressing and bottling machinery that are not cleaned regularly and sufficiently, however, the biggest source of Brett contamination is from used barrels.

Brett is rarely found in juice or fermenting wines, so its levels, if present, must be below what would be detected by peering through a microscope or plating for S. cerevisiae, i.e. less than 10⁶ cells/ml. After fermentation is complete, Brett can be found in high concentrations. This yeast is very alcohol tolerant, with some strains able to use ethanol as sole carbon source. Often Brett is detected in barreled wine by the smell. It can produce several very “aromatic” substances, variously described as “Band-aid”, “horsey”, “sweaty”, and “wet-dog-in-a-phone-booth”. One of the compounds, 4-ethyl guaiacol, can have a rather pleasant spicy aroma, but it is usually overpowered by the strong-smelling, 4-ethyl phenol, or may not be considered a desirable or appropriate character for the wine. Brett is also involved in the formation of biogenic amines, which can cause headaches in wine drinkers.

Monitoring wine for Brett is not an easy process for most wineries. While there are currently several methods available for detection of Brett, because it is very slow-growing (1-2 weeks on agar plates), spoilage can and often does occur before the winemaker is aware of contamination. Rapid methods are available, but are prohibitively expensive for large-scale use and require considerable investment in equipment, time, and expertise. A simple, inexpensive method that could detect Brett directly from wine in a few hours would give the winemaker an “early warning” tool in his or her campaign against Brett. For those who like a little Brett in their winemaking for stylistic reasons, this method could serve as a monitoring device to keep the levels of Brett in check.

Conventional methods in current use for assaying yeasts are commonly based on expensive and time consuming methods. An example of this is mass spectrometry, as described in the published patent application no. 2003/0,162,221 A1, with inventors G. Bader et. al., published on Aug. 28, 2003. Other methods include traditional culturing of yeasts, as described, for example, in U.S. Pat. No. 7,108,980, which issued on Sept. 19, 2006 to inventors J. Hyldig-Nielsen et al.

The most widely used method for monitoring spoilage yeasts such as Brett in wine is agar plating which can take up to 10-14 days. Alternative methods such as RT-PCR are available but are too expensive and complex to install even in large wineries, and require highly trained people to operate.

Immunoassay-based methods for pathogen detection are rapidly replacing microbiological methods in medicine. This trend is related to technical advances in immunochemistry that allow the development of such methods to be more rapid, specific, and lower in cost than traditional methods. The application of immunochemistry to the testing for wine spoilage yeast and bacteria has, for the most part, not been attempted using the new generation of highly sensitive immunoassays that are currently available. Another modern technique, DNA sequencing, is used in early phases of development to detect spoilage organisms, such as Brett. DNA sequencing uses “primers” for gene amplification to identify a specific species. Very little DNA is needed for such amplification to take place, and therefore, even a miniscule amount of DNA can result in a false positive. This issue confines DNA sequencing to very clean, laboratory environments overseen by highly skilled technicians. There are methods to control contamination and estimate the number of infectious cells; however, these require a sterile lab environment, highly trained people, and expensive equipment. These requirements result in a per-test result that may be too high for all but the most premium wines, and may take days to send out samples and receive results. In contrast, immunoassays are very resistant to contamination and only require minimum training of staff.

Accordingly, there is an unmet need to adapt rapid, sensitive and specific immunoassay techniques to detection of yeast in fermented beverages.

### SUMMARY OF THE INVENTION

A method is disclosed for detecting yeast or other microorganism, such as preferably Brettanomyces, in a fermented beverage sample, such as preferably, wine. The methods comprises the steps of:

a) spotting the sample onto a biochip, comprising dry PVDF adhered to a rigid support, such as preferably Miragene's proprietary Z-GripT™ substrate;

b) contacting the biochip with an antibody specific for the yeast or other microorganism, wherein the antibody is conjugated to an enzyme, such as preferably alkaline phosphatase;

c) adding a substrate for the enzyme, wherein the enzyme catalyzes a calorimetric change in the substrate, such that a visible spot develops on the biochip; and

d) optional scanning of the biochip to determine the density of the spot.

In a variation to the above described method, the contacting step (b) comprises contacting the biochip with a primary antibody specific for the yeast, wherein the primary antibody is from a first species; and then contacting the biochip with a secondary antibody which is conjugated to an enzyme, wherein the secondary antibody is from a second species and configured to specifically bind the primary antibody.

A kit for detecting yeast in wine is disclosed in accordance with another preferred embodiment of the present invention. The kit comprises: a) a biochip, comprising dry PVDF adhered to a rigid support; b) a primary antibody specific for the yeast, wherein the primary antibody is conjugated to alkaline phosphatase; and c) a developing reagent comprising BCIP/NBT substrate.

### BRIEF DESCRIPTION OF THE DRAWINGS

FIG. 1 shows Brettanomyces strains in wine.

FIG. 2 shows an increasing concentration of Brett on a developed assay chip in accordance with preferred embodiments of the present invention.

FIG. 3 shows the scanned image of the above chip illustrated in FIG. 2.

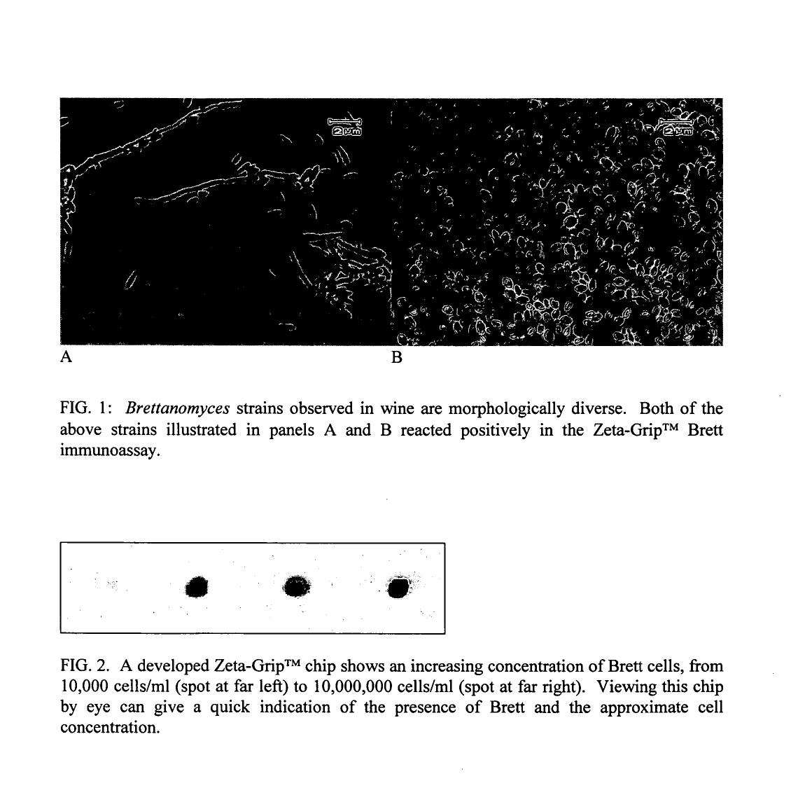

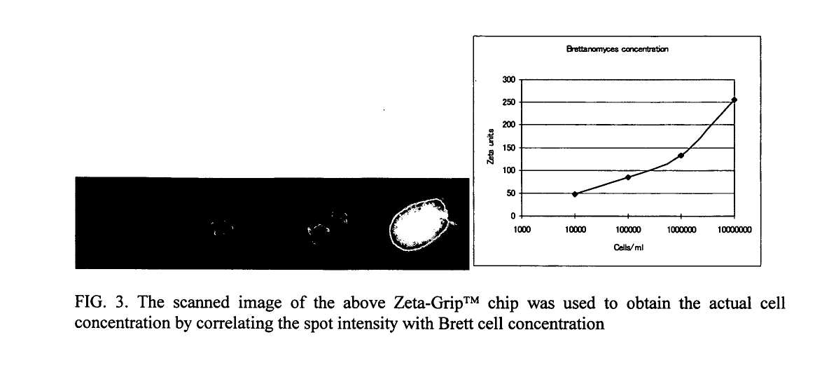

### DETAILED DESCRIPTION OF THE PREFERRED EMBODIMENT

The assay for Brett detection is an immunoassay-based test, which can be performed within a few hours without any special training or equipment, at a cost of only a few dollars per test. This assay is based on a proprietary substrate called Zeta-Grip™, which together with proprietary reagents may be provided in kit form for use by even the smallest wineries. The assay may be adapted for other wine microorganisms, e.g. the early fermentation spoilage yeast, Hanseniaspora or Kloeckera, the bottled wine spoilage yeast, Zygosaccharomyces, and various lactic acid bacteria, such as Pediococcus and Lactobacillus. In one preferred embodiment, the assay may be adapted to detect several different yeasts and bacteria on one assay chip. The method can be readily adapted for assaying microorganisms in other fermented beverages, such as beer, or non-alcoholic beverages, such as milk, water, or other beverages.

Applicants’ assay method requires almost no special equipment, is low-cost, and results can be obtained in less than three hours. While this technique was initially developed and validated for medical applications, the Applicants developed a rapid, low-cost test for Brett using the new Zeta-Grip™ technology.

Miragene's Zeta-Grip™ substrate provides a novel approach by utilizing a membrane, which in the past has always been used in a ‘wet’ state, in a ‘dry’ state and adhered to a rigid surface. This allows proteins to be spotted onto the substrate, allowed to dry, and then interact with other proteins.

In one preferred embodiment, samples of wine are spotted onto the substrate, allowed to dry, and then tested for Brett by allowing the wine sample to react with an anti-Brett antibody conjugated with the alkaline phosphotase enzyme. Any antibody specific for Brett can be used. In another embodiment, the primary antibody could be unconjugated (e.g., a rabbit anti-Brett antibody), and a secondary enzyme-conjugated antibody (e.g., an Alk-phos-conjugated goat anti-rabbit IgG), specific for the primary antibody could be used. In any event, the result, if Brett is present in the wine, is a colored spot which is usually visible by eye or can be detected and quantified with an ordinary flatbed scanner.

In another embodiment, a biochip is provided in a multi-well format. Different samples of wine are spotted, e.g., using an eye dropper or any suitable liquid dispensing device, onto the biochip next to identifier numbers. The numbers can be used to identify each sample from the barrel or bottle from which it originated. In one embodiment, the identifier numbers may be bar-coded. In another embodiment, the wine may be concentrated by centrifugation prior to spotting.

After spotting between 1 and 500, and more preferable, between 1 and 100, and most preferably between 1 and 48 samples of wine, or any other fermented liquid, on a biochip, several solutions (provided in kit) are added to the well—detailed below, which then are shaken on a small, portable shaker. The final step is the addition of a developer reagent. The developer creates a purple spot in any sample that contains Brett. The intensity of the spot is proportional to the amount of Brett in the sample. In order to determine the exact concentration of Brett, the biochip is scanned using a typical, flatbed computer scanner, and a computer program is used to calculate the amount of Brett contamination. Anyone with the skills involved in winemaking should be able to conduct the test easily and reliably.

Z-GRIP™ Substrate

The Z-GRIP™ substrate preferred for use as a biochip in the yeast assays described herein are disclosed in co-pending U.S. application Ser. No. 10/376,351 filed on Feb. 27, 2003; the entire disclosure of which is incorporated herein by reference. Most traditional protein studies have involved wet chemistries and porous membranes such as the polymer polyvinylidene fluoride (PVDF), which is widely used in techniques such as western blot. Experiments were carried out to evaluate if proteins could be immobilized on the surface of a membrane such as PVDF when it is in a dry state.

PVDF was adhered to a glass support using an inert double-sided adhesive microfilm. Samples, such as Brett, were spotted onto the dry surface of the PVDF and after drying were allowed to interact with antibodies with a conjugated secondary antibody. Results demonstrated that the laminated substrate overcame the many problems encountered with existing substrates. In addition, the opaque nature of the membrane together with the chemical detection systems described herein allowed the interactions to be detected and analyzed on a low-cost flatbed scanner using light in the visual wavelength spectrum.

In one embodiment, the present invention provides a Z-GRIP™ array substrate that can be used in a dry state to immobilize sample. A hydrophobic membrane is included that immobilizes proteins in a reduced surface area with minimal diffusion across the membrane. The laminated membrane adheres to a glass surface with a double-sided inert adhesive microfilm, and preferably includes a protective polymer layer over the PVDF substrate surface.

In one embodiment, the present invention can be used with multiple conjugated secondary antibodies such as Alkaline Phosphatase (AP), Biotin Protein A, or enzyme labels such as HRP or fluorescent dyes etc. In one preferred embodiment, the present invention optionally includes a barcode for test and/or sample identification and data archiving. In another embodiment, the primary antibody is conjugated to the detection chemistry, so that no secondary antibody is needed, and development and visualization requires only addition of the enzyme substrate.

In one embodiment, the present invention provides an array with very little background noise. More specifically, the background noise for the Z-GRIP™ PVDF-coated glass slide using the alkaline phosphatase (AP) reaction for detection of proteins, visualized using a conventional flatbed scanner, is less than about 100 lumens. More preferably, the background on the Z-GRIP™ developed as above is between about 50 and 0 lumens. Most preferably, the background is from about 15 to 0 lumens. Similarly little to no background is seen when a fluorescent dye is used for protein detection on the Z-GRIP™ PVDF-coated glass slide and imaged using a fluorescent scanner. In contrast, typical backgrounds seen using commercial protein substrates, e.g., slides with epoxy surface chemistries, are above 200 lumens and usually in the 300 to 400 lumen range.

Maximum signal intensities for the Z-GRIP™ PVDF-coated glass slide in accordance with a preferred embodiment of the present invention using the Alkaline phosphatase reaction for detecting proteins and a conventional flatbed scanner for quantifying spot densities (otherwise referred to herein as “protein imaging”), analyzed using commercial imaging software (e.g., Adobe PHOTOSHOP®) are about 15,000 to 25,000 lumens. Maximum signal intensities for the Z-GRIP™ substrate using fluorescent detection chemistries and a fluorescent scanner are usually about 25,000 lumens. Although epoxy substrates also produce maximum signal intensities of about 25,000 lumens with either AP or fluorescent detection, because of the relatively high background levels seen with epoxy slides, the Z-GRIP™ PVDF-coated biochips have approximately 10-fold greater total dynamic range and signal-to-noise ratios than other protein substrates. Moreover, in accordance with a preferred embodiment of the present invention, background for any detection chemistry on a PVDF-coated rigid support is less than about 1% of the maximal signal intensity, and more preferably, in the range of about 0.1% to about 1%, and most preferably about 0.1% (e.g., 25 lumens background/25,000 lumens max signal).

In addition to the advantages discussed above with regard to the higher signal-to-noise ratio seen with a preferred embodiment of the present invention, the Z-GRIP™ PVDF-coated rigid supports also generate enhanced assay sensitivity because the hydrophobic PVDF surface facilitates superior sample spotting/density than the hydrophilic surface chemistries typically used for protein arrays (See e.g., Salinaro et al. WO 01/61042 which teaches the criticality of using a hydrophilic surface for biomolecular arrays). As a result of the hydrophobic nature of PVDF, samples spotted onto the dry PVDF surface tend to stay in high density, very discrete spots, which do not spread and diffuse through the polymeric substrate. Thus, the sample density is relatively high compared to samples spotted onto hydrophilic and/or wetted substrates. As a result of the high density, the concentration of yeast in the sample does not become limiting on the subsequent detection reactions (e.g., conjugated primary or secondary antibody binding). Where spots have spread in hydrophilic substrates, the relative sample concentrations are much lower and become limiting on the detection reactions. Consequently, the sensitivity seen using the Z-GRIP™ hydrophobic surface chemistry was observed to be approximately 1000-fold greater than sensitivities obtained with the same samples and detection reactions on a hydrophilic surface.

Binding Interaction

The binding interaction between the primary antibodies and the yeast antigens spotted on the test strip focuses on the association between two substances or molecules. Antibody is considered to “bind” to yeast if, after incubation of the antibody (usually in solution or suspension) with or on the test strip for a period of time (usually 5 minutes or more, for instance 10 minutes, 20 minutes, 30 minutes, 60 minutes, 90 minutes, 120 minutes or more), a detectable amount of the antibody associates with a yeast antigen to such an extent that it is not removed by being washed with a relatively low stringency buffer (e.g., 100 mM KCl). In some applications, e.g., where nonspecific binding is elevated, higher stringency buffers may be used.

Washing can be carried out, for instance, at room temperature, but other temperatures (either higher or lower) can also be used. Antibodies will bind different antigens to different extents, and the term “bind” encompasses both relatively weak and relatively strong interactions. Thus, some binding will persist after the strip is washed in a higher salt buffer (e.g., 500 mM or 1000 mM KCl).

Quantification of the binding pattern can be carried out using any of several existing techniques, including scanning the signals into a computer for calculation of relative density of each spot. Quantitation methodology is discussed in greater detail below.

The prior art is replete with methods for detecting reactions and interactions between two molecules. One possible embodiment of the present invention may be modified by one skilled in the art to accommodate the various detection methods known in the art. The exact detection method chosen by one in the art will depend on several factors, including the amount of biological sample available, the biological sample type, the stability of the biological sample, the stability of the reactant and the affinity between the reactant and analyte. Moreover, as discussed above, depending on the detection methods chosen, it may be required to modify the reactant and biological sample.

While these techniques are well known in the art, examples of a few of the detection methods which could be utilized to practice one possible embodiment of the present invention are briefly described below.

Immunoassays

There are many types of immunoassays known in the art. The most common type of immunoassay are competitive and non-competitive heterogeneous assays such as enzyme-linked immunosorbent assays (ELISA). In immunoassays the reactant is an antigen. In a noncompetitive ELISA, unlabeled antigen (e.g., yeast) is bound to a solid phase such as a PVDF-coated solid support. Primary antibody is combined with antigens bound to the substrate and are allowed to bind to the antigens forming immune complexes. After immune complexes have formed, excess antibody is removed and the support is washed to remove nonspecifically bound antibodies. If the primary antibodies are conjugated to detection chemistries (e.g., enzyme), then the enzyme linked to the antibody catalyzes a reaction which converts added substrate into product—which preferably produces a visible spot. If the primary antibody is not conjugated to an enzyme, the immune complexes are then reacted with an appropriate enzyme-labeled anti-immunoglobulin (secondary antibody). Anti-immunoglobulins recognize bound antibodies, but not antigens. Anti-immunoglobulins specific for antibodies of different species, including human, are well known in the art and commercially available from Sigma Chemical Company, St. Louis, Mo. and Santa Cruz Biotechnology, Santa Cruz, Calif. After a second wash step, the enzyme substrate is added. The enzyme linked to the secondary antibody catalyzes a reaction which converts substrate into product. Typically, the reaction product is colored and thus measured spectrophotometrically using UVNIS technology and equipment well known in the art.

Sandwich or capture assays can also be used to identify and quantify immune complexes. Sandwich assays are a mirror image of non-competitive ELISAs, antibodies are bound to the solid phase and antigen in the blood is measured (analyte). These assays are particularly useful in detecting antigens that are present at low concentrations having multiple epitopes. This technique requires excess primary antibody (e.g., anti-Brett) to be attached to a solid phase, such as the Z-Grip™ substrate. The bound antibody is then incubated with the wine sample and yeast in the sample are allowed to form immune complexes with the bound antibody. The immune complex is incubated with an enzyme-linked secondary antibody which recognizes the same or a different epitope on the yeast as the bound antibody. Hence, enzyme activity is directly proportional to the amount of yeast in the sample. See Kemeny, D M, and S. J. Challacombe (eds), ELISA and Other Solid Phase Immunoassays, John Wiley & Sons, Chichester, 1988 which is incorporated by reference.

Typical enzymes that can be linked to secondary antibodies include horseradish peroxidase, glucose oxidase, glucose-6-phosphate dehydrogenase, alkaline phosphates, (β-D-galactosidase and urease. Secondary antigen-specific antibodies linked to various enzymes are commercially available from, for example, Sigma Chemical Company, St Louis, Mo. and Amersham Life Sciences, Arlington Height, Ill.

Fluorescent immunoassays can also be employed when practicing the method of one possible embodiment of the present invention. Fluorescent immunoassays are similar to ELISAs except that the enzyme is substituted for fluorescent compounds called fluorophores or fluorochromes. These compounds have the ability to absorb energy from incident light and reemit the energy as light of a longer wavelength and lower energy. Fluorescein and rhodamine, usually in the form of isothiocyanates which can be readily coupled to reactants and antibodies are most commonly used in the art. See Stites, D. P. et al., Basic and Clinical Immunology; Appleton & Lange, east Norwalk, Conn. (1994) hereby incorporated by reference. Fluorescein absorbs light of 490 to 495 nm in wavelength and emits green light at 520 nm in length. Tetramethylrhodamine absorbs light of 550 nm in wavelength and emits red light at 580 nm in length.

Phycobiliproteins isolated from algae, porphyrins, and chlorophylls which all fluoresce at approximately 600 nm are also being used in the art. See Hemmila, I., Fluoroimmunoassays and Immunofluorometric Assays. Clin Chem, 31: 359 (1985) and U.S. Pat. No. 4,542,104 to Stryer et al. hereby incorporated by reference. Phycobiliproteins and derivative are commercially available under the names R-phycoerythrin (PE) and Quantum Red™ from for example, Sigma Chemical Company, St. Louis, Mo.

In addition, Cy-conjugated secondary antibodies and reactants are useful in immunoassays and are commercially available. Cy-3, for example, is maximally excited at 554 run and emits light of between 568 and 574 rim. Cy-3 is more hydrophilic than other fluorophores and thus has less of a tendency to bind nonspecifically or aggregate. Cy-conjugated include Cy-2, Cy-3, and Cy-5 are commercially available from Amersham Life Sciences, Arlington Height, Ill.

The techniques described above for detecting analytes in a sample are only exemplary of the many techniques that could be employed with one possible embodiment of the present invention. One skilled in the art will appreciate that one possible embodiment of the present invention can be modified to accommodate many other techniques including radioimmune assays (RIA), biotin-antibody conjugated assays, time resolved fluorescence, colloidal gold conjugates assays, ferritin conjugates assays, western blotting, variable number of tandem repeats assays, short tandem repeat assays and sex specific assays using probes for detecting human Y-specific regions.

Digitizing the Results

Once interactions between the yeast and antibodies have been identified and quantified, the signals may be digitized for storage and to facilitate analysis. Regardless of whether a fluorescent dye (quantified using a fluorescence scanner) or other colorometric signal, e.g., Alkaline Phophatase—BCIP/NBT developing reagent (quantified using a conventional flatbed scanner), the spot density can be digitized and analyzed using any of a variety of commercially available imaging and densitometry software, such as for example, Adobe PHOTOSHOP®, Array Vision, Spotware, etc. An identifier number or conventional bar code on the test strip preferably identifies the source of the sample (e.g., barrel number X) used to generate the digital profile.

It will be appreciated by one skilled in the art that other methods of developing, imaging, storing and accessing the microarray spot densities may be employed.

Results

Preliminary tests were conducted on Brett strains Vin 1 (1) and Vin 8 (A), which are distinct morphologically and physiologically. Decreasing concentrations of Brett were used to test the sensitivity of the assay. Tests have shown promising results with a visible reaction to Brett down to a concentration of approximately 1,000 cells/ml. The purple spots were very clear due to a strong immunoreaction as well as the absence of background noise. From about 10⁷ cells/ml to about 10,000 cells/ml, the signal showed a linear correlation making the prediction of Brett concentration reliable. Cross-reactivity with other yeasts may be minimized if desired by purifying the anti-Brett antibody. There was negligible cross-reaction with bacteria and foreign contamination from the tester. In preferred variations, the assay sensitivity may be increased to detection limits of less than 1000 cells/ml, less than 500 cells/ml, less than 100 cells/ml, and most preferably less than about 10 cells/ml. Conventional immunoassay techniques may be applied to affect the enhanced sensitivity.

Brett spoilage can occur at concentrations as low as 1000 cells/ml. Thus, in accordance with one preferred embodiment, an effective assay preferably detects a few hundred Brett cells/ml. Such an outcome would allow a winemaker to maintain his or her winemaking style and avoid the damage that Brett can inflict on a wine. Applicants have demonstrated that the disclosed assay has the potential to do exactly this.

In preferred variations, the test biochip may be blocked with a protein solution, e.g., casein (0.1-10%, preferably about 2%) to reduce nonspecific binding. Primary antibody may be incubated with the chip for between 5 min and 12 hr, more preferable, between about 30 min and 1 hr. The primary antibody may be a conjugated or unconjugated antibody specific for the particular yeast being assayed (e.g., anti-Brett). The primary antibody may be a polyclonal or monoclonal antibody of any species, preferably mammalian (e.g., mouse, rabbit, goat, human, etc.). Antibodies that specifically bind to Brett can be made by conventional procedures. The chips are preferably washed after contacting with the antibody, e.g., 1-5×, preferably about 3× in PBS before addition of developer (e.g., enzyme substrate) or the secondary anti-primary (species-specific) IgG conjugated to enzyme, (e.g., Pierce Biochemicals, Rockford Ill, Goat anti human IgG Alkaline Phosphatase Conjugated Product #31310). After the second antibody binding incubation, the arrays may be washed again as above, and a developing reagent is added (e.g., 1 ml×BCIP/NBT, Pierce Biochemicals). After about 5-60 min, preferably about 15 min, the biochips are washed again, allowed to dry, and scanned in a commercial flatbed scanner.

Detailed descriptions of the preferred embodiment are provided herein, above. It is to be understood, however, that the present invention may be embodied in various forms. Therefore, specific details disclosed herein are not to be interpreted as limiting, but rather as a basis for the claims and as representative basis for teaching one skilled in the art to employ the present invention in virtually any appropriately detailed system, structure or manner.

What is claimed is:

1. A method for detecting yeast in a sample, comprising:
a) Spotting said sample on a test strip, said strip comprising dry PVDF adhered to a rigid support;
b) Contacting said strip with an antibody specific for the yeast, wherein the antibody is conjugated to an enzyme;
c) Adding a substrate to said strip, said substrate enabling the development of a visible spot on said strip; and
d) Scanning said strip to determine the density of said visible spot.

2. The method of claim 1, wherein the antibody is a microorganism specific monoclonal or polyclonal antibody.

3. The method of claim 1, wherein the enzyme is alkaline phosphatase.

4. The method of claim 1, wherein the yeast is Brettanomyces.

5. The method of claim 1, wherein the antibody is conjugated to a fluorescence molecule such as horseradish peroxidase and detected by a fluorescence scanner.

6. The method of claim 1, wherein the antibody is conjugated to a bioluminescent molecule such as luciferase, and detected by a photomultiplier tube.

7. The method in claim 1, wherein the contaminating whole cell is a eukaryote or prokaryote, including yeast, bacteria and mammalian cells.

8. A method for detecting contaminating whole cells in a sample, comprising:
a) Spotting said sample on a test strip, said strip comprising dry PVDF adhered to a rigid support;
b) Contacting said strip with an antibody specific for the yeast, wherein the antibody is conjugated to an enzyme;
c) Adding a substrate to said strip, said substrate enabling the development of a visible spot on said strip; and
d) Scanning said strip to determine the density of said visible spot.

9. The method of claim 8, wherein the contaminating whole cells are spoilage microorganisms in fermented beverages.

10. The method of claim 8, wherein the contaminating whole cells are Brettanomyces, Zygosaccharomyces or other wine and beer spoilage microorganisms.

11. The method of claim 8, wherein the contaminating whole cells are spoilage microorganisms in carbonated beverages.

12. The method of claim 8, wherein the contaminating whole cells are spoilage microorganisms in milk, water and other non-fermented or carbonated solutions.

13. The method of claim 8, wherein the yeast is Brettanomyces.

14. The method of claim 8, wherein said sample is a fermented beverage.

15. The method for claim 8, wherein the assay platform is a biochip consisting of a dry PVDF membrane adhered to a rigid support with an inert polymer binder.

16. A method for detecting yeast in a sample, comprising:
a) spotting said sample on a test strip, comprising dry PVDF adhered to a ridged support;
b) contacting said strip with a primary antibody specific for the yeast, wherein the primary antibody is from a first species;
c) contacting said strip with a secondary antibody conjugated to an enzyme, wherein the second antibody is specific for the primary antibody, wherein the secondary antibody is from a second species;
d) adding a substrate, for said enzyme, wherein said enzyme catalyzes a colorimetric change in the substrate, such that a visible spot develops on said strip; and
e) scanning said strip to determine the density of the visible spot.

17. A kit for detecting yeast in wine, comprising:
a) a test strip, comprising dry PVDF adhered to a ridged support;
b) a primary antibody specific for the yeast, wherein the primary antibody is conjugated to alkaline; and
c) a developing reagent comprising BCIP/NBT substrate.
